(19) United States
(12) Patent Application Publication
(10) Pub. No.: US 2024/0216399 A1
(43) Pub. Date: Jul. 4, 2024

(54) CANNABINOID COMPOSITION AND METHOD OF USING THE SAME

(71) Applicants: Adan Arriaga, Jasper, TX (US); Saulo Arriaga, Jasper, TX (US); Joshua Harpel, San Antonio, TX (US)

(72) Inventors: Adan Arriaga, Jasper, TX (US); Saulo Arriaga, Jasper, TX (US); Joshua Harpel, San Antonio, TX (US)

(21) Appl. No.: 18/603,705

(22) Filed: Mar. 13, 2024

Related U.S. Application Data
(63) Continuation of application No. PCT/US2022/076323, filed on Sep. 13, 2022.
(60) Provisional application No. 63/243,361, filed on Sep. 13, 2021.

(51) Int. Cl. A61K 31/00; A23L 2/44; A23L 2/54; A61K 9/00; A61K 9/107

(52) U.S. Cl. CPC A61K 31/658; A23L 2/44; A23L 2/54; A61K 9/0095; A61K 9/1075

(57) ABSTRACT

A cannabinoid composition is provided that comprises a liquid carrier and a first lipid carrier comprising at least one cannabinoid, the first lipid carrier having a particle size from 10 nm to 100 nm. The cannabinoid composition further includes a second lipid carrier comprising at least one cannabinoid, the second lipid carrier having a particle size from 200 nm to 600 nm.

### CROSS-REFERENCE TO RELATED APPLICATIONS

This application is a continuation of International Patent Application Serial No. PCT/US2022/076323 filed on Sep. 13, 2022 and entitled, “CANNABINOID COMPOSITION AND METHOD OF USING THE SAME”, which claims priority to U.S. Patent Application Ser. No. 63/243,361, filed on Sep. 13, 2021, the contents and disclosures of which are incorporated by reference herein in their entireties for all purposes.

### TECHNICAL FIELD

This disclosure relates in general to therapeutic or recreational compositions, and more particularly to cannabinoid compositions and methods of using the same.

### BACKGROUND

Cannabinoids, such as delta-9-tetrahydrocannabinol (THC) or cannabidiol (CBD), have been shown to reduce or treat the effects of various diseases or disorders. For example, cannabinoids have been shown to reduce or treat the effects of anorexia associated with weight loss in patients with AIDS, nausea and vomiting induced by chemotherapy, relieve pain (e.g., spasticity and neuropathic pain due to multiple sclerosis or pain in adult patients with cancer), comatose after severe traumatic brain injury, atopic dermatitis, weight loss, and nicotine addiction among others.

Cannabinoids are typically administered by inhalation or oral administration. Subjects seeking therapeutic or recreational effects associated with cannabinoids must take multiple administrations via inhalation or oral administration to achieve an initial release of the cannabinoid and then a subsequent release to extend the effects thereafter. Currently, there is a need for a cannabinoid composition that provides an initial release and an extended release in a single dosage of the cannabinoid composition.

### SUMMARY OF THE DISCLOSURE

The compositions and methods of the present disclosure addresses at least the above-mentioned drawbacks by providing a cannabinoid composition that provides an initial release and an extended release in a single dosage of the cannabinoid composition. In some embodiments, the provided cannabinoid composition includes a first lipid carrier selectively sized to provide an initial release of at least one cannabinoid to a subject's lymphatic system and a second lipid carrier selectively sized to provide at least one cannabinoid via an extended release to a subject's hepatic system.

According to one embodiment, the present disclosure provides a cannabinoid composition comprising a liquid carrier. The composition further comprises a first lipid carrier encapsulating at least one cannabinoid, where the first lipid carrier has a particle size from 10 nm to 100 nm. The composition further comprises a second lipid carrier encapsulating at least one cannabinoid, where the second lipid carrier has a particle size from 200 nm to 600 nm.

According to one embodiment, the present disclosure provides a method of manufacturing a beverage comprising a cannabinoid composition. The method includes replacing at least a portion of the oxygen in a container with carbon dioxide. The method further includes receiving a cannabinoid composition in the container, where the cannabinoid composition includes a liquid carrier, a first lipid carrier comprising at least one cannabinoid, the first lipid carrier having a particle size from 10 nm to 100 nm, and a second lipid carrier comprising at least one cannabinoid, the second lipid carrier having a particle size from 200 nm to 600 nm. The method further includes receiving a sterilant and water in the container, and capping the container.

Other technical advantages will be readily apparent to one skilled in the art from the following figures, descriptions, and claims. Moreover, while specific advantages have been enumerated above, various embodiments may include all, some, or none of the enumerated advantages.

### BRIEF DESCRIPTION OF THE DRAWINGS

For a more complete understanding of the present disclosure and its advantages, reference is now made to the following description, taken in conjunction with the accompanying drawings, in which:

FIG. 1 is a schematic illustration of a cannabinoid composition, according to certain embodiments;

FIG. 2 illustrates a flow chart for a method of manufacturing a beverage containing a cannabinoid composition, according to certain embodiments.

### DETAILED DESCRIPTION OF THE DISCLOSURE

Current cannabinoid compositions that provide a therapeutic or recreational effect require multiple administrations to achieve an initial release of the cannabinoid and then a subsequent release to extend the effect at a point thereafter. The compositions and methods of the present disclosure address the above-mentioned drawbacks by providing a cannabinoid composition that provides an initial release and an extended release in a single dosage of the cannabinoid composition. The cannabinoid composition may release at least one cannabinoid to a subject's lymphatic system via an initial release, and release at least one cannabinoid to a subject's hepatic system via an extended release.

To facilitate a better understanding of the present disclosure, the following examples of certain embodiments are given. In no way should the following examples be read to limit, or define, the scope of the disclosure. Embodiments of the present disclosure and its advantages may be best understood by referring to the included FIGURES, where like numbers are used to indicate like and corresponding parts.

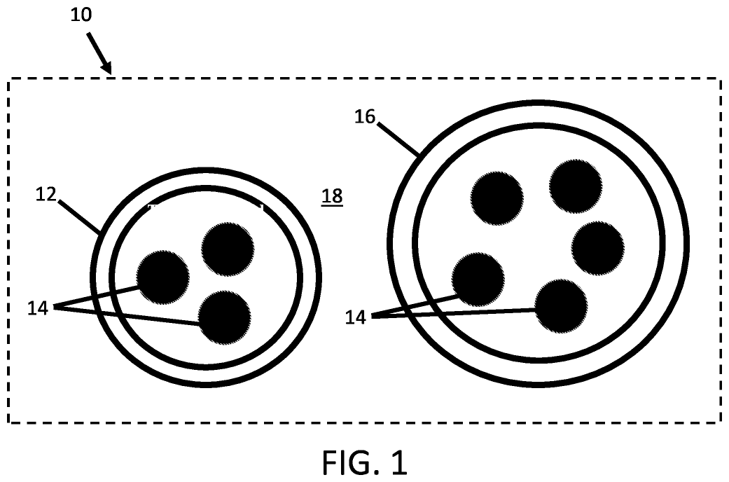

FIG. 1 is a schematic illustration of a cannabinoid composition 10 according to an embodiment of the present disclosure. The cannabinoid composition 10 includes a first lipid carrier 12 that comprises at least one cannabinoid 14, and a second lipid carrier 16 that comprises at least one cannabinoid 14. The first lipid carrier 12 and the second lipid carrier 16 may be suspended in a liquid carrier 18. In some embodiments, the first lipid carrier 12 and the second lipid carrier 16 improve the bioavailability of the at least one cannabinoid 14, which are typically poorly soluble in the liquid carrier 18.

As used herein, the term “lipid carrier” may refer to a lipid-based vehicle that entrains or encapsulates the at least one cannabinoid 14. Suitable lipid carriers 12, 16 include, but are not limited to, nanoemulsions, liposomes, micelles, nanostructured lipid carriers, solid lipid nanoparticles, or combinations thereof. Exemplary lipids include, but are not limited to, phospholipids, glyceryl stearate, glyceryl palmitostearate, glyceryl trilaurate, cholesterol, glyceryl dibehenate, cetyl palmitate, stearic acid, tripalmitin, caprylic/capric triglycerides (e.g., medium chain triglycerides), lauroyl polyoxylglycerides, monoacylglycerols, soya lecithin, squalene, sunflower oil, corn oil, or combinations thereof.

As used herein, the term “cannabinoid” may refer to a chemical compound that has direct or indirect activity on a cannabinoid receptor in a subject's brain. Exemplary cannabinoids include, but are not limited to, delta-9-tetrahydrocannabidinol (THC), delta-9-tetrahydrocannabinol-C4 (THC-C4), delta-9-tetrahydrocannabiorcol (THC-C1), delta-9-tetrahydrocannabiorcolic acid (THCA-C1), delta-9-tetrahydrocannabinolic acid-C4 (THCA-C4), delta-9-tetrahydrocannabivarin (THCV), delta-9-tetrahydrocannabivarinic acid (THCVA), delta-9-tetrahydrocannabinolic acid A (THCA-A), delta-9-tetrahydrocannabinolic acid B (THCA-B), delta-9-tetrahydrocannabiphorol (THCP), cannabidiol (CBD), cannabidiol monomethylether (CBDM), cannabidiolic acid (CBDA), cannabidivarinic acid (CBDVA), cannabidiorcol (CBD-C1), cannabidivarin (CBDV), cannabidiphorol (CBDP), cannabicyclolic acid (CBLA), cannabicyclol (CBL), cannabicyclovarin (CBLV), cannabichromene (CBC), cannabichromevarinic acid (CBCVA), cannabichromenic acid (CBCA), cannabichromevarin (CBCV), cannabielsoin (CBE), cannabielsoin acid A (CBEA-A), cannabielsoic acid B (CBEA-B), 10-Ethoxy-9-hydroxy-delta-6a-tetrahydrocannabinol, 8,9-dihydroxy-delta-6a-tetrahydrocannabinol, cannabitriol (CBT), cannabitriolvarin (CBTV), cannabigerol (CBG), cannabigerovarin (CBGV), cannabigerovarinic acid (CBGVA), cannabigerol monomethylether (CBGM), cannabigerolic acid monomethylether (CBGAM), cannabigerolic acid (CBGA), cannabinolic acid (CBNA), cannabinodiol (CBND), cannabinodivarin (CBVD), cannabinol (CBN), cannabiorcool (CBN-C1), cannabivarin (CBV), cannabinol methylether (CBNM), cannabinol-C2 (CBN-C2), cannabinol-C4 (CBN-C4), delta-8-tetrahydrocannabinol (Δ8-THC), delta-8-tetrahydrocannabinolic acid (Δ8-THCA), 10-Oxo-delta-6a-tetrahydrocannabinol (OTHC), cannabichromanon (CBCF), cannabifuran (CBF), cannabiglendol Delta-9-cis-tetrahydrocannabinol (cis-THC), cannbicitran (CBT), dehydrocannabifuran (DCBF), tryhydroxy-delta-9-tetrahydrocannabinol (triOH-THC), cannabiripsol (CBR), or combinations thereof.

As used herein, the term “subject” may refer to any organism to which a cannabinoid composition is used in accordance embodiments of the present disclosure e.g., for experimental, diagnostic, prophylactic, recreational, and/or therapeutic purposes. Typical subjects include animals (e.g., mammals such as mice, rats, rabbits, non-human primates, and humans; insects; worms; etc.). In some embodiments, a subject may be suffering from, and/or susceptible to a disease, disorder, and/or condition (e.g., weight loss, nausea and vomiting induced by chemotherapy, pain such as spasticity and neuropathic pain due to multiple sclerosis or pain in adult patients with cancer, comatose after severe traumatic brain injury, atopic dermatitis, nicotine addiction, etc.).

As used herein, the term “liquid carrier” may refer to solvents or dispersion media that are pharmaceutically acceptable to the subject. Exemplary liquid carriers 18 include, but are not limited to, aqueous solutions or dispersions (e.g., sterile aqueous solutions or dispersions) that optionally contain, for example, water, cell culture medium, acylglycerols, polyol (e.g., glycerol, propylene glycol, liquid polyethylene glycol), or combinations thereof.

In some embodiments, the cannabinoid composition 10 includes from 60% to 95% (w/w) of a liquid carrier 18, based on a total mass of the cannabinoid composition 10. In some embodiments, the cannabinoid composition 10 includes at least 60% (w/w) of a liquid carrier 18, or at least 65%, at least 70%, at least 75%, at least 80% to less than 85%, less than 90%, or less than 95% (w/w) based on a total mass of the cannabinoid composition 10.

In some embodiments, the cannabinoid composition 10 includes from 0.1% to 5% (w/w) of a first lipid carrier 12, based on a total mass of the cannabinoid composition. In some embodiments the cannabinoid composition 10 includes at least 0.1% (w/w) of a first lipid carrier 12, or at least 1%, at least 1.5%, at least 2%, at least 2.5% to less than 3%, less than 3.5%, less than 4%, or less than 5% (w/w) based on a total mass of the cannabinoid composition 10.

In some embodiments, the first lipid carrier 12 is sized for an initial release of the at least one cannabinoid 14 to the subject. In some embodiments, the first lipid carrier 12 is selectively sized to deliver the at least one cannabinoid 14 to a subject's lymphatic system following oral administration (e.g., administration by mouth of a composition). In some embodiments, the first lipid carrier 12 has a particle size from 10 nm to 100 nm. In some embodiments, the first lipid carrier has a particle size of at least 10 nm, at least 15 nm, at least 20 nm, at least 25 nm, at least 30 nm, or at least 35 nm to less than 40 nm, less than 45 nm, less than 50 nm, less than 55 nm, less than 60 nm, less than 65 nm, less than 70 nm, less than 75 nm, less than 80 nm, less than 85 nm, less than 90 nm, less than 95 nm, or less than 100 nm. The particle size of the first lipid carrier 12 may be determined using known methods such as direct microscopy, optical microscopy, LASER particle analysis, conductivity or Coulter counter techniques.

In some embodiments, the cannabinoid composition 10 includes from 0.1% to 10% (w/w) of a second lipid carrier 18, based on a total mass of the cannabinoid composition. In some embodiments, the cannabinoid composition 10 includes at least 0.1% (w/w) of a second lipid carrier 18, or at least 0.1% (w/w) of a second lipid carrier 18, or at least 1%, at least 1.5%, at least 2%, at least 2.5%, at least 3%, at least 3.5%, at least 4%, at least 4.5% to less than 5%, less than 6%, less than 7%, less than 8%, less than 9%, or less than 10% (w/w), based on a total mass of the cannabinoid composition 10.

In some embodiments, the second lipid carrier 16 is sized for an extended release of the at least one cannabinoid 14 to the subject. In some embodiments, the second lipid carrier 16 is selectively sized to deliver the at least one cannabinoid 16 to a subject's hepatic system following oral administration. In some embodiments, the second lipid carrier 16 has a particle size of at least 200 nm to 600 nm. In some embodiments, the second lipid carrier 16 has a particle size of at least 200 nm, at least 225 nm, at least 250 nm, at least 275 nm, at least 300 nm, at least 325 nm, at least 350 nm, to at least 375 nm or less than 400 nm, less than 425 nm, less than 450 nm, less than 475 nm, less than 500 nm, less than 525 m, less than 550 nm, less than 575 nm, or less than 600 nm. The particle size of the second lipid carrier 16 may be determined using known methods such as direct microscopy, optical microscopy, LASER particle analysis, conductivity or Coulter counter techniques.

In some embodiments, the cannabinoid composition 10 further comprises a surfactant. The surfactant may act to reduce the surface tension of the liquid carrier and/or may act as an emulsifier in the cannabinoid composition 10. Exemplary surfactants include, but are not limited to, polysorbates (e.g., polysorbate 20 or 80), poloxamers (e.g., poloxamer 188 or 407), polyaxamine, macrogol-15-hydrooxystearate, polyoxiethylene stearate, phosphatidyl choline, polyoxyl castor oil, or combinations thereof. In some embodiments, the cannabinoid composition 10 includes from 0.1% to 10% (w/w) of a surfactant, based on a total mass of the cannabinoid composition. In some embodiments, the cannabinoid composition 10 includes at least 0.1% (w/w) of a surfactant, or at least 1%, at least 1.5%, at least 2%, at least 2.5%, at least 3%, at least 3.5%, at least 4%, at least 4.5% to less than 5%, less than 6%, less than 7%, less than 8%, less than 9%, or less than 10% (w/w), based on a total mass of the cannabinoid composition 10.

In some embodiments, the cannabinoid composition 10 comprises a buffer. Exemplary buffers include, but are not limited to, citric acid, tartaric acid, lactic acid, salts thereof or combinations thereof. In some embodiments, the cannabinoid composition 10 has a pH that ranges from 3 to 3.7. In some embodiments, the cannabinoid composition 10 has a pH of at least 3, at least 3.1, at least 3.2, at least 3.3 to less than 3.4, less than 3.5, less than 3.6, or less than 3.7. In some instances, maintaining a pH between 3 to 3.7 helps to stabilize the at least one cannabinoid in the cannabinoid composition 10, and may reduce the likelihood, or prevent, the at least one cannabinoid from degrading in the cannabinoid composition 10. In some embodiments, the cannabinoid composition 10 includes a preservative (e.g., benzoates, sorbates, salts thereof or combinations thereof).

Without being bound to any particular theory, the inventors have found that delivery of cannabinoids to the hepatic system is less efficient than delivery to the lymphatic system. To facilitate in ameliorating this shortcoming, the cannabinoid composition 10 may include a higher mass ratio of second lipid carriers 16 relative to first lipid carriers 12. In some embodiments, a mass ratio of the second lipid carrier 16 relative the first lipid carrier 12 is in an amount from 3:1 to 10:1 in the cannabinoid composition 10, i.e., (a total mass of second lipid carrier): (a total mass of first lipid carrier). In some embodiments, the mass ratio of the second lipid carrier 16 relative to the first lipid carrier 12 is at least 3:1, at least 4:1, at least 5:1, at least 6:1 to less than 7:1, less than 8:1, less than 9:1, or less than 10:1.

In some embodiments, the cannabinoid composition 10 includes from 1 mg to 400 mg of at least one cannabinoid 14. In some embodiments, the cannabinoid composition 10 includes at least 1 mg of at least one cannabinoid 14, at least 2 mg, at least 3 mg, at least 4 mg, at least 5 mg, at least 10 mg, at least 15 mg, at least 20 mg, at least 25 mg, at least 30 mg at least 35 mg to less than 40 mg, less than 50 mg, less than 100 mg, less than 200 mg, less than 300 mg, or less than 400 mg of the least one cannabinoid.

In some embodiments, the first lipid carrier 12 includes from 1 mg to 50 mg of the at least one cannabinoid 14. In some embodiments, the first lipid carrier 12 includes at least 1 mg of at least one cannabinoid 14, at least 2 mg, at least 3 mg, at least 4 mg, at least 5 mg, at least 10 mg, at least 15 mg to less than 20 mg, less than 25 mg, less than 30 mg, less than 35 mg, less than 40 mg, less than 45 mg, or less than 50 mg of the at least one cannabinoid 14.

In some embodiments, the second lipid carrier 16 includes from 10 mg to 350 mg of the at least cannabinoid 14. In some embodiments the second lipid carrier 16 includes at least 10 mg of the at least one cannabinoid 14, at least 15 mg, at least 20 mg, at least 25 mg, at least 30 mg, at least 35 mg, at least 40 mg, at least 45 mg, at least 50 mg to less than 100 mg, less than 150 mg, less than 200 mg, less than 250 mg, less than 300 mg, or less than 350 mg.

In some embodiments, the first lipid carrier 12 and the second lipid carrier 16 include cannabinoids 14 that are the same. In other embodiments, the first lipid carrier 12 includes at least one cannabinoid 14 that is distinct from the at least one cannabinoid 14 in the second lipid carrier 16.

In some embodiments, the cannabinoid composition is formulated for oral administration in a solid dosage form (e.g., capsules, cachets, pills, tablets, lozenges using a flavored basis such as sucrose and acacia or tragacanth, powders, gummies, sublingual strips, infused candy, and granules). In some embodiments, the cannabinoid composition 10 is formulated in the solid dosage form by mixing the cannabinoid composition 10 with fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol, glycerol monostearate, and non-ionic surfactants; absorbents, such as kaolin and bentonite clay; lubricants, such as talc, calcium stearate, magnesium stearate, solid polyethylene glycols, sodium lauryl sulfate, or combinations thereof; or coloring agents.

Tablets may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made in a suitable machine in which a mixture of the powdered composition is moistened with an inert liquid diluent.

Tablets, and other solid dosage forms of the cannabinoid composition 10 of the present disclosure, such as dragees, capsules, pills, and granules, may optionally be scored or prepared with coatings and shells, such as enteric coatings and other coatings well known in the pharmaceutical-formulating art. They may be sterilized by, for example, filtration through a bacteria-retaining filter, or by incorporating sterilizing agents in the form of sterile solid compositions that can be dissolved in sterile water, or some other sterile injectable medium immediately before use.

In some embodiments, the cannabinoid composition 10 is formulated in a liquid dosage form (e.g., emulsions, microemulsions, solutions, suspensions, syrups, beverages, spray, and elixirs). In addition to the components of the cannabinoid composition 10, the liquid dosage form may include inert ingredients such as for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, or mixtures thereof. Besides inert ingredients, the liquid dosage form may include wetting agents, emulsifying agents, sweetening, flavoring (e.g., juices or concentrate), vitamins, minerals, coloring, perfuming or preservative agents. Beverages may be carbonated. Suspensions containing the cannabinoid composition 10 may also include suspending agents as, for example, ethoxylated isostearyl alcohols, polyoxyethylene sorbitol and sorbitan esters, microcrystalline cellulose, aluminum metahydroxide, bentonite, agar-agar and tragacanth, or mixtures thereof.

The cannabinoid composition 10 may be formulated in a topical dosage form (e.g., ointments, pastes, creams, lotions, and gels). The ointments, pastes, creams and gels may contain, in addition to the cannabinoid composition 10, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

In some embodiments, the cannabinoid composition 10 can be administered to the subject through oral administration or topical administration.

In some embodiments, the cannabinoid composition 10 may be manufactured using, for example, a hot high-pressure homogenization technique, a cold high-pressure homogenization technique, a microemulsion based technique, a double emulsion based technique, a membrane contractor technique, a phase inversion technique, a solvent emulsification-diffusion technique, a solvent emulsification-evaporation technique, or a solvent injection technique.

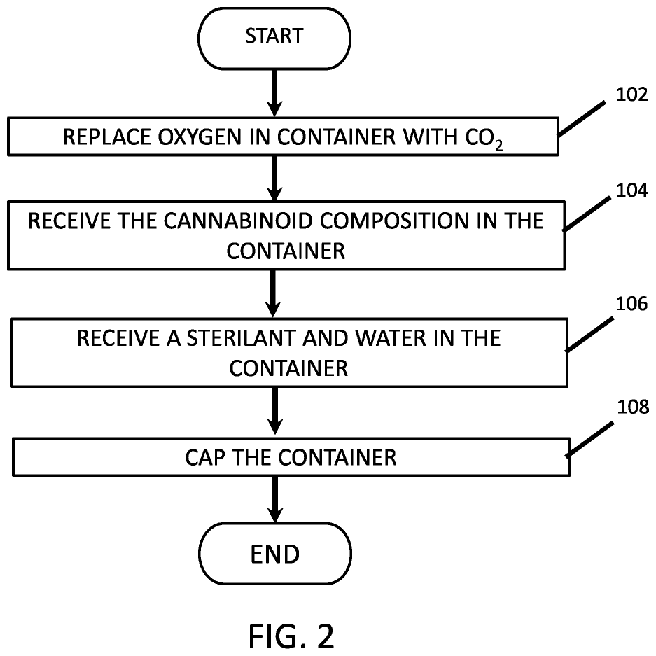

Referring to FIG. 2, the present disclosure provides a method 100 of manufacturing a beverage comprising the cannabinoid composition 10. The inventors have found that one of the challenges associated with manufacturing a beverage containing a cannabinoid is that pasteurization of beverage ingredients (e.g., concentrate or fruit juices) tends to damage or degrade cannabinoids. To address this issue, the present disclosure provides a method 100 of manufacturing a beverage comprising the cannabinoid composition 10 without pasteurizing the beverage. In some embodiments, the method 100 begins at operation 102, which includes providing a container to receive a beverage therein, and replacing at least a portion of a container with carbon dioxide. In some embodiments, carbon dioxide is used to pushed out the oxygen from the container. At operation 104, the method 100 further includes receiving the cannabinoid composition 10 in the container. At operation 106, the method further includes receiving a sterilant and water in the container to fill the container to a desired level. The sterilant may comprising dimethyl dicarbonate. Dimethyl dicarbonate may be used for preventing microbial spoilage in the beverage. One of the advantages of dimethyl dicarbonate is it may be used to prevent spoilage without having to pasteurize (i.e., heat to beverage to temperatures that degrade cannabinoids).

Operation 106 may further include adding other ingredients, such as flavoring agents, buffers, sweetening agents, vitamins, minerals, coloring agents, perfuming agents, salts, thickeners, anti-foaming agents, and preservative agents. In some embodiments, the pH of the beverage ranges from 3 to 3.7, which further helps to protect against spoiling microorganisms. In some embodiments, the sterilant is sufficient to protect against spoiling microorganisms and the beverage may be substantially free or entirely free of preservative agents. As used herein, the term “substantially free” refers to a beverage having less than 0.3% (w/w), or less than 0.2% (w/w), or less than 0.1% (w/w) of a preservative agent, based on a toal mass of the beverage. The cannabinoid composition 10, sterilant, water and other ingredients may be transported to the container using any technique, e.g., a filler in a bottling process, a pump, etc. At operation 108, the method 100 includes capping the container to seal the beverage within the container.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, functions, operations, or steps, any of these embodiments may include any combination or permutation of any of the components, elements, functions, operations, or steps described or illustrated anywhere herein that a person having ordinary skill in the art would comprehend. Furthermore, reference in the appended claims to an apparatus or system or a component of an apparatus or system being adapted to, arranged to, capable of, configured to, enabled to, operable to, or operative to perform a particular function encompasses that apparatus, system, component, whether or not it or that particular function is activated, turned on, or unlocked, as long as that apparatus, system, or component is so adapted, arranged, capable, configured, enabled, operable, or operative.

What is claimed is:

1. A cannabinoid composition comprising:
a liquid carrier;
a first lipid carrier comprising at least one cannabinoid, the first lipid carrier having a particle size from 10 nm to 100 nm; and
a second lipid carrier comprising at least one cannabinoid, the second lipid carrier having a particle size from 200 nm to 600 nm.

2. The cannabinoid composition of claim 1, wherein the first lipid carrier has a size from 15 nm to 85 nm, from 20 nm to 60 nm, from 25 nm to 55 nm, or from 30 to 40 nm.

3. The cannabinoid composition of claim 1, wherein the second lipid carrier has a size from 300 nm to 500 nm, from 350 nm to 550 nm, or from 375 nm to 525 nm.

4. The cannabinoid composition of claim 1 comprising from 60% to 95% (w/w) of the liquid carrier, based on a total mass of the cannabinoid composition.

5. The cannabinoid composition of claim 1 comprising from 0.1% to 5% (w/w) of the first lipid carrier, based on a total mass of the cannabinoid composition.

6. The cannabinoid composition of claim1 comprising from 0.1% to 10% (w/w) of the second lipid carrier, based on a total mass of the cannabinoid composition.

7. The cannabinoid composition of claim 1 further comprising a surfactant.

8. The cannabinoid composition of claim 7 comprising from 1% (w/w) to 10% (w/w) of the surfactant, based on a total mass of the composition.

9. The cannabinoid composition of claim 1 further comprising a buffer.

10. The cannabinoid composition of claim 9 comprising from 0.1% to 5% (w/w) of the buffer, based on a total mass of the cannabinoid composition.

11. The cannabinoid composition of claim 9, wherein the pH of the composition ranges from 3 to 3.7.

12. The cannabinoid composition of claim 1, wherein the second lipid carrier is present in a ratio to the first lipid carrier in an amount of at least 3:1.

13. The cannabinoid composition of claim 12, wherein the second lipid carrier is present in a mass ratio to the first lipid carrier in an amount from 3:1 to 10:1.

14. The cannabinoid composition of claim 1, wherein the first lipid carrier comprises cannabinoids that are distinct from the cannabinoids in the second lipid carrier.

15. The cannabinoid composition of claim 1, wherein the first lipid carrier and the second lipid carrier contain the same cannabinoids.

16. The cannabinoid composition of claim 1 included in a solid dosage form or a liquid dosage form.

17. A method comprising administering the cannabinoid composition of claim 1 to a subject through oral administration or topical administration.

18. A method of manufacturing a beverage comprising a cannabinoid composition, the method comprising:
replacing at least a portion of the oxygen in a container with carbon dioxide;
receiving a cannabinoid composition in the container, the cannabinoid composition comprising a liquid carrier, a first lipid carrier comprising at least one cannabinoid, the first lipid carrier having a particle size from 10 nm to 100 nm, and a second lipid carrier comprising at least one cannabinoid, the second lipid carrier having a particle size from 200 nm to 600 nm;
receiving a sterilant and water in the container; and
capping the container.

19. The method of claim 18, wherein the sterilant is dimethyl dicarbonate.

20. The method of claim 18, wherein the first lipid carrier has a size from 15 nm to 85 nm, from 20 nm to 60 nm, from 25 nm to 55 nm, or from 30 to 40 nm; and wherein the second lipid carrier has a size from 300 nm to 500 nm, from 350 nm to 550 nm, or from 375 nm to 525 nm.
